(19) United States
(12) Patent Application Publication
(10) Pub. No.: US 2008/0125428 A1
(43) Pub. Date: May 29, 2008

(54) Cancer Treatment Method

(76) Inventor: David Rusnak, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/574,331

(22) PCT Filed: Aug. 25, 2005
(86) PCT No.: PCT/US05/30148

§ 371 Date: Feb. 27, 2007

Related U.S. Application Data
(60) Provisional application No. 60/605,404, filed on Aug. 27, 2004.

(51) Int. Cl. A61K 31/5377; A61P 35/00; A61K 31/517

(52) U.S. Cl. 514/234.5; 514/266.24

(57) ABSTRACT

The present invention relates to a method of treating cancer in a mammal by administration of 4-quinazolinamines and at least one additional EGFR and/or erbB-2 inhibitor. In particular, the method relates to methods of treating cancers by administration of N-{3-chloro-4-[(3-fluorobenzyl)oxy]phenyl}-6-[5-({[2-(methanesulphonyl)ethyl]amino}methyl)-2-furyl]-4-quinazolinamine and salts and solvates thereof in combination with at least one additional EGFR and/or erbB-2 inhibitor.

### BACKGROUND OF THE INVENTION

The present invention relates to a method of treating cancer in a mammal by administration of 4-quinazolinamines, which are dual inhibitors of EGFR and erbB-2, in combination with at least one other erbB family inhibitor. In particular, the method relates to methods of treating cancers which are mediated by the tyrosine kinases EGFR and/or erbB2 by administration of N-{3-chloro-4-[(3-fluorobenzyl)oxy]phenyl}-6-[5-({[2-(methanesulphonyl)ethyl]amino}methyl)-2-furyl]-4-quinazolinamine and salts and solvates thereof along with EGFR inhibitors gefitinib and/or erlotinib.

Effective chemotherapy for cancer treatment is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways is cellular signaling from growth factor receptors at the cell surface to the nucleus (Crews and Erikson, 1993), in particular, cellular signalling from the growth factor receptors of the erbB family.

There is significant interaction among the erbB family that regulates the cellular effects mediated by these receptors. Six different ligands that bind to EGFR include EGF, transforming growth factor, amphiregulin, heparin binding EGF, betacellulin, and epiregulin (Alroy & Yarden, FEBS Letters, 410:83-86, 1997; Burden & Yarden, Neuron, 18: 847-855, 1997; Klapper et al., Proc Natl Acad Sci, 4994-5000, 1999). Heregulins, another class of ligands, bind directly to HER3 and/or HER4 (Holmes et al., Science, 256:1205, 1992; Klapper et al., 1997, Oncogene, 14:2099-2109; Peles et al., Cell, 69:205, 1992). Binding of specific ligands induces homo- or heterodimerization of the receptors within members of the erbB family (Carraway & Cantley, Cell, 78:5-8, 1994; Lemmon & Schlessinger, Trends Biochem Sci, 19:459-463, 1994). In contrast with the other erbB receptor members, a soluble ligand has not yet been identified for HER2, which seems to be transactivated following heterodimerization. The heterodimerization of the erbB-2 receptor with EGFR (erbB-1), HER3 (erbB-3), and HER4 (erbB-4) is preferred to homodimerization (Klapper et al., 1999; Klapper et al., 1997). Receptor dimerization results in binding of ATP to the receptor's catalytic site, activation of the receptor's tyrosine kinase, and autophosphorylation on C-terminal tyrosine residues. The phosphorylated tyrosine residues then serve as docking sites for proteins such as Grb2, Shc, and phospholipase C, that, in turn, activate downstream signaling pathways, including the Ras/MEK/Erk and the PI3K/Akt pathways, which regulate transcription factors and other proteins involved in biological responses such as proliferation, cell motility, angiogenesis, cell survival, and differentiation (Alroy & Yarden, 1997; Burgering & Coffer, Nature, 376:599-602, 1995; Chan et al., Ann Rev Biochem, 68:965-1014, 1999; Lewis et al., Adv Can Res, 74:49-139, 1998; Liu et al., Genes and Dev, 13:786-791, 1999; Muthuswamy et al., Mol & Cell Bio, 19, 10:6845-6857, 1999; Riese & Stern, Bioessays, 20:41-48, 1998).

Several strategies including monoclonal antibodies (Mab), immunoconjugates, anti-EGF vaccine, and tyrosine kinase inhibitors have been developed to target the erbB family receptors and block their activation in cancer cells (reviewed in (Sridhar et al., Lancet, 4, 7:397-406, 2003)). Because erbB2-containing heterodimers are the most stable and preferred initiating event for signaling, interrupting both erbB2 and EGFR simultaneously is an appealing therapeutic strategy. A series of 6-furanylquinazoline dual erbB-2/EGFR tyrosine kinase inhibitors that possess efficacy in pre-clinical models for cancer have been synthesized (Cockerill et al., Biorg Med Chem Lett, 11:1401-1405, 2001; Rusnak et al., Can Res, 61:7196-7203, 2001a; Rusnak et al., Mol Can Ther, 1:85-94, 2001b). GW572016 is a 6-furanylquinazoline, orally active, reversible dual kinase inhibitor of both EGFR and erbB2 kinases (Rusnak et al., 2001b). In human xenograft studies, GW572016 has shown dose-dependent kinase inhibition, and selectively inhibits tumor cells overexpressing EGFR or erbB2 (Rusnak et al., 2001b; Xia et al., Oncogene, 21:6255-6263, 2002).

Combination therapy is rapidly becoming the norm in cancer treatment, rather than the exception. Oncologists are continually looking for anti-neoplastic compounds which when utilized in combination provides a more effective and/or enhanced treatment to the individual suffering the effects of cancer. Typically, successful combination therapy provides improved and even synergistic effect over monotherapy.

The present inventors have now identified novel cancer treatment methods which include administration of N-{3-chloro-4-[(3-fluorobenzyl)oxy]phenyl}-6-[5-({[2-(methanesulphonyl)ethyl]amino}methyl)-2-furyl]-4-quinazolinamine (GW572016) as well as salts and/or solvates thereof in combination with an additional inhibitor of EGFR and/or erbB-2.

### SUMMARY OF THE INVENTION

In a first aspect of the present invention, there is provided a method of treating breast cancer in a mammal, comprising: administering to said mammal therapeutically effective amounts of

(i) a compound of formula (I″)

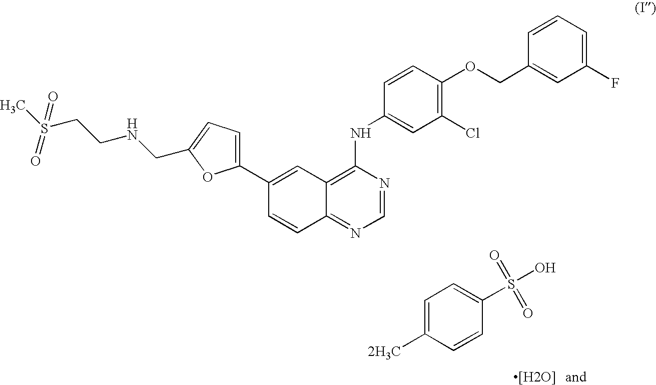

(ii) gefitinib or salts or solvates thereof.

In a second aspect of the present invention, there is provided a cancer treatment combination, comprising: therapeutically effective amounts of

(i) a compound of formula (I″)

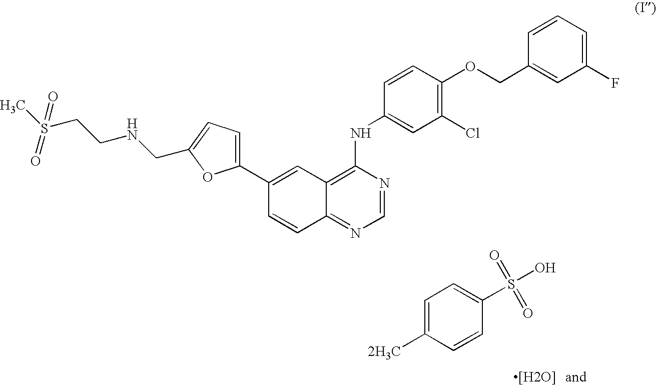

(ii) gefitinib or salts or solvates thereof.

In a third aspect of the present invention, there is provided a method of treating breast cancer in a mammal, comprising: administering to said mammal therapeutically effective amounts of

(i) a compound of formula (I″)

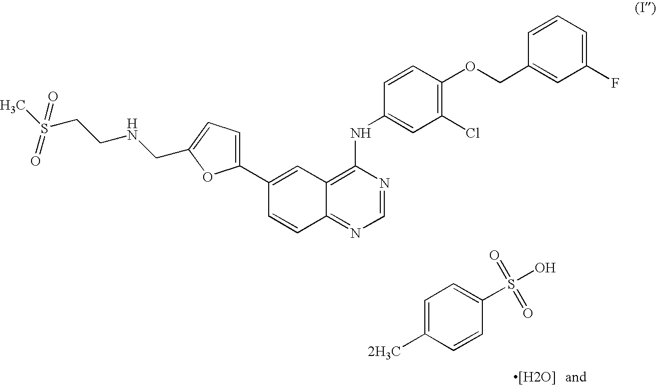

(ii) erlotinib or salts or solvates thereof.

In a fourth aspect of the present invention, there is provided a cancer treatment combination, comprising: therapeutically effective amounts of

(i) a compound of formula (I″)

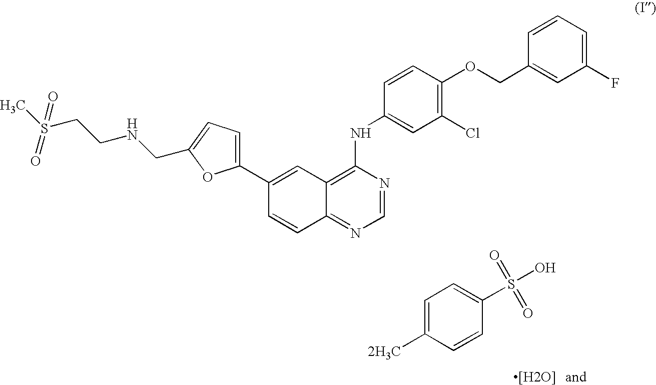

(ii) erlotinib or salts or solvates thereof.

In a fifth aspect of the present invention, there is provided a method of treating breast cancer in a mammal, comprising: administering to said mammal therapeutically effective amounts of

(i) a compound of formula (I″)

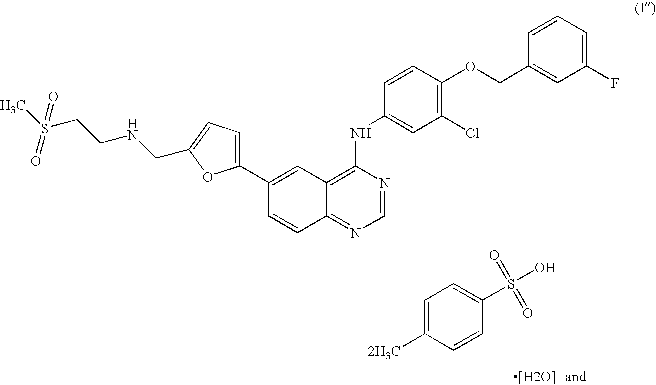

(ii) gefitinib and/or erlotinib or salts or solvates thereof.

In a sixth aspect of the present invention, there is provided a cancer treatment combination, comprising: therapeutically effective amounts of

(i) a compound of formula (I″)

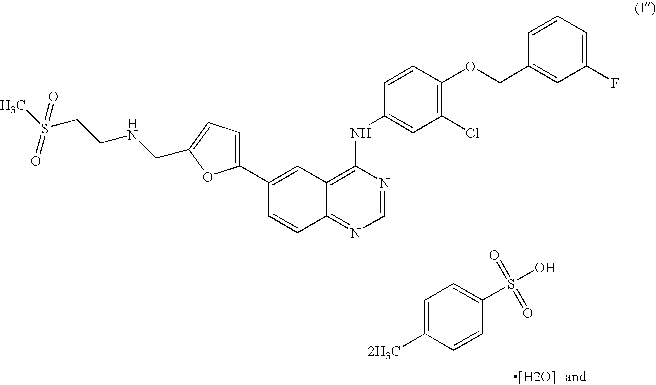

(ii) gefitinib and/or erlotinib or salts or solvates thereof.

### BRIEF DESCRIPTION OF THE DRAWINGS

FIG. 1 depicts a graph showing the relative growth of A459 lung carcinoma cells when dosed with varying concentrations of GW572016F (lapatinib), gefitinib or both.

FIG. 2 depicts a graph showing the relative growth of BT474 breast carcinoma cells when dosed with varying concentrations of GW572016F (lapatinib), gefitinib or both.

FIG. 3 depicts a graph showing the relative growth of Colo205 colon carcinoma cells when dosed with varying concentrations of GW572016F (lapatinib), gefitinib or both.

FIG. 4 depicts a graph showing the relative growth of HN5 head and neck carcinoma cells when dosed with varying concentrations of GW572016F (lapatinib), gefitinib or both.

FIG. 5 depicts a graph showing the relative growth of MDA468 breast carcinoma cells when dosed with varying concentrations of GW572016F (lapatinib), gefitinib or both.

FIG. 6 depicts a graph showing the relative growth of A459 lung carcinoma cells when dosed with varying concentrations of GW572016F (lapatinib), erlotinib or both.

FIG. 7 depicts a graph showing the relative growth of MDA468 breast carcinoma cells when dosed with varying concentrations of GW572016F (lapatinib), erlotinib or both.

FIG. 8 depicts an additional graph showing the relative growth of MDA468 breast carcinoma cells when dosed with varying concentrations of GW572016F (lapatinib), erlotinib or both in a second experiment.

FIG. 9 depicts a graph showing the relative growth of H1975 lung carcinoma cells when dosed with varying concentrations of GW572016F (lapatinib), erlotinib or both.

FIG. 10 depicts an additional graph showing the relative growth of H1975 lung carcinoma cells when dosed with varying concentrations of GW572016F (lapatinib), erlotinib or both in a second experiment.

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As is well known in the art, cancers or tumors are frequently metastatic, in that a first (primary) locus of cancerous tumor growth spreads to one or more anatomically separate sites. As used herein, reference to “a tumor” in a subject includes not only the primary tumor, but metastatic tumor growth as well. In a like manner reference to cancer or cancer treatment includes primary and metastatic cancer and treatment of the primary cancer as well as metastatic cancerous sites.

“EGFR”, also known as “erbB-1”, and “erbB-2” are protein tyrosine kinase transmembrane growth factor receptors of the erbB family. Protein tyrosine kinases catalyse the phosphorylation of specific tyrosyl residues in various proteins involved in the regulation of cell growth and differentiation (A. F. Wilks, Progress in Growth Factor Research, 1990, 2, 97-111; S. A. Courtneidge, Dev. Supp. I, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401). The ErbB family of type I receptor tyrosine kinases includes ErbB1 (also known as the epidermal growth factor receptor (EGFR or HER1)), erbB2 (also known as Her2), erbB3, and erbB4. These receptor tyrosine kinases are widely expressed in epithelial, mesenchymal, and neuronal tissues where they play a role in regulating cell proliferation, survival, and differentiation (Sibilia and Wagner, Science, 269: 234 (1995); Threadgill et al., Science, 269: 230 (1995)). Increased expression of wild-type erbB2 or EGFR, or expression of constitutively activated receptor mutants, transforms cells in vitro (Di Fiore et al., 1987; DiMarco et al, Oncogene, 4: 831 (1989); Hudziak et al., Proc. Natl. Acad. Sci. USA., 84:7159 (1987); Qian et al., Oncogene, 10:211 (1995)). Increased expression of erbB2 or EGFR has been correlated with a poorer clinical outcome in some breast cancers and a variety of other malignancies (Slamon et al., Science, 235: 177 (1987); Slamon et al., Science, 244:707 (1989); Bacus et al, Am. J. Clin. Path, 102:S13 (1994)).

As used herein, the term “solvate” refers to a complex of variable stoichiometry formed by a solute (in this invention, compounds of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

In one embodiment of the present invention, there is provided a method of treating a susceptible cancer in a mammal, comprising: administering to said mammal therapeutically effective amounts of

(i) a compound of formula (I″)

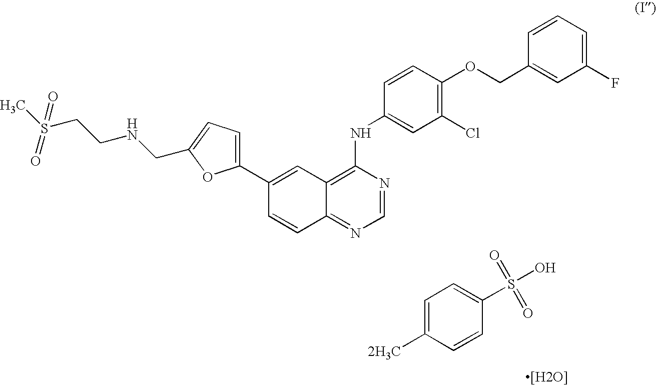

(ii) gefitinib or salts or solvates thereof; and/or

(iii) erlotinib or salts or solvates thereof.

As recited above the present invention is directed to cancer treatment methods which includes administration of N-{3-chloro-4-[(3-fluorobenzyl)oxy]phenyl}-6-[5-({[2-(methanesulphonyl)ethyl]amino}methyl)-2-furyl]-4-quinazolinamine (GW572016) as well as salts and/or solvates thereof in combination with another EGFR and/or erbB-2 inhibitor.

The methods of cancer treatment disclosed herein includes administering a compound of formula (I):

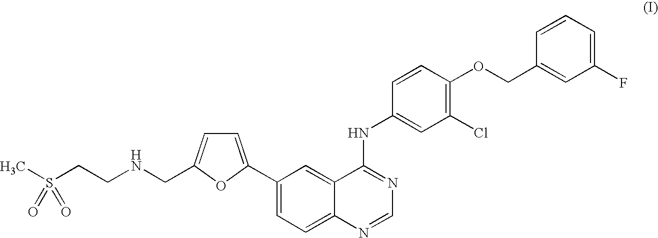

or salts or solvates thereof.

In another embodiment, the compound is a compound of formula (I′) which is the ditosylate salt of the compound of formula (I) or anhydrate or hydrate forms thereof. The ditosylate salt of the compound of formula (I) has the chemical name N-{3-chloro-4-[(3-fluorobenzyl)oxy]phenyl}-6-[5-({[2-(methanesulphonyl)ethyl]amino}methyl)-2-furyl]-4-quinazolinamine (GW572016) ditosylate and is also known as lapatinib.

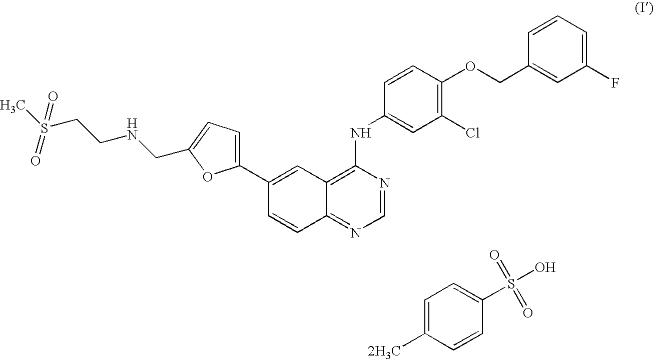

In one embodiment, the compound is the anhydrous ditosylate salt of the compound of formula (I′). In another embodiment, the compound is a compound of formula (I″) which is the monohydrate ditosylate salt of the compound of formula (I′).

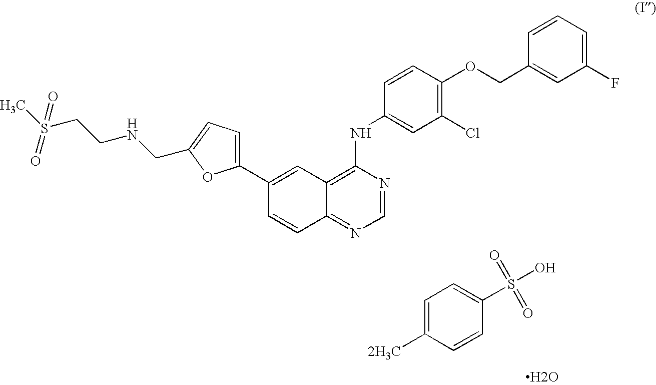

The free base, HCl salts, and ditosylate salts of the compound of Formula (I) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (I″) is presented following in Scheme 1.

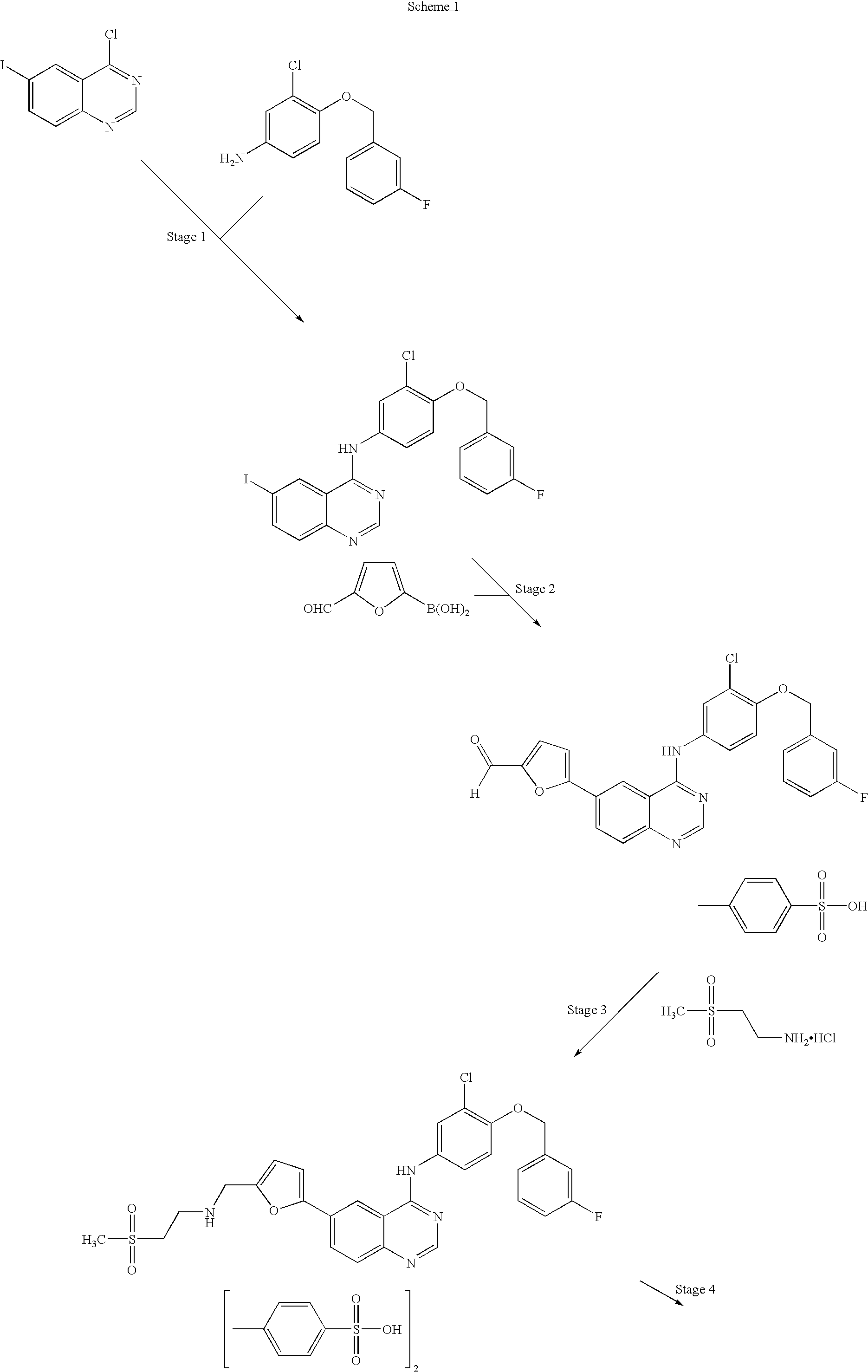

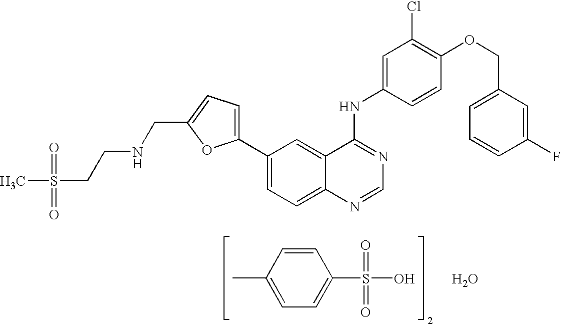

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

In one embodiment, the cancer treatment method is a method of treating breast cancer wherein the compound of formula (I″) is administered with gefitinib.

Gefitinib, 4-quinazolinamine,N-(3-chloro-4-fluorophenyl)-7-methoxy-6-[3-4-morpholin)propoxy]; is commercially available as tablets as IRESSA®. Gefitinib is represented by the structure of formula (II)

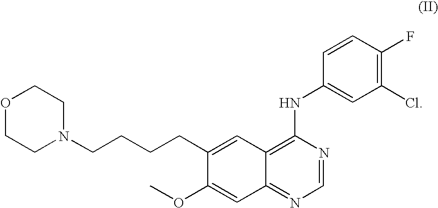

### Gefitinib is an EGFR inhibitor that is indicated as monotherapy for the treatment of patients with locally advanced or metastatic non-small-cell lung cancer after failure of both platinum-based and docetaxel chemotherapies.

The free base, HCl salts, and diHCl salts of the compound of Formula (II) may be prepared according to the procedures of International Patent Application No. PCT/GB96/00961, filed Apr. 23, 1996, and published as WO 96/33980 on Oct. 31, 1996.

In one embodiment, the cancer treatment method is a method of treating non small cell lung cancer wherein the compound of formula (I″) is administered with gefitinib.

In one embodiment, the cancer treatment method is a method of treating colorectal cancer wherein the compound of formula (I″) is administered with gefitinib.

In one embodiment, the cancer treatment method is a method of treating head and neck cancer wherein the compound of formula (I″) is administered with gefitinib.

In another embodiment, the cancer treatment method is a method of treating breast cancer wherein the compound of formula (I″) is administered with erlotinib.

Erlotinib, N-(3-ethynylphenyl)-6,7-bis(2-methoxyethoxy)-4-quinazolinamine; is commercially available as tablets as TARCEVA™. Erlotinib is represented by the structure of formula (III)

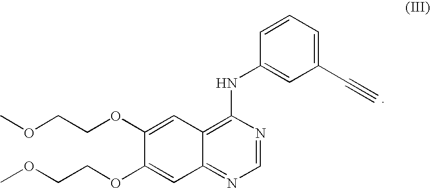

In one embodiment, erlotinib is in the salt form: erlotinib hydrochloride. Erlotinib is an EGFR inhibitor that is indicated for the treatment of patients with locally advanced or metastatic non-small-cell lung cancer after failure of at least one prior chemotherapy regiment.

The free base and HCl salts of the compound of Formula (III) may be prepared according to the procedure of Example 20 of U.S. Pat. No. 5,747,498, issued May 5, 1998.

In another embodiment, the cancer treatment method is a method of treating non small cell lung cancer wherein the compound of formula (I″) is administered with erlotinib.

In another embodiment, the cancer treatment method is a method of treating colorectal cancer wherein the compound of formula (I″) is administered with erlotinib.

In another embodiment, the cancer treatment method is a method of treating head and neck cancer wherein the compound of formula (I″) is administered with erlotinib.

In one embodiment, the cancer treatment method is a method of treating breast cancer wherein the compound of formula (I″) is administered with gefitinib and erlotinib.

In one embodiment, the cancer treatment method is a method of treating non small cell lung cancer wherein the compound of formula (I″) is administered with gefitinib and erlotinib.

In one embodiment, the cancer treatment method is a method of treating colorectal cancer wherein the compound of formula (I″) is administered with gefitinib and erlotinib.

In one embodiment, the cancer treatment method is a method of treating head and neck cancer wherein the compound of formula (I″) is administered with gefitinib and erlotinib.

Combination therapies according to the present invention thus include the administration of the compound of formula (I″) as well as use of at least one other EGFR and/or erbB-2 inhibitor, preferably gefitinib. Such combination of agents may be administered together or separately and, when administered separately this may occur simultaneously or sequentially in any order, both close and remote in time. The amounts of the compound of formula (I″) and the other pharmaceutically active agent(s) and the relative timings of administration will be selected in order to achieve the desired combined therapeutic effect.

Accordingly, in another aspect of the present invention is a pharmaceutical combination, preferably a cancer treatment combination, which includes a compound of formula (I″) and gefitinib. In another aspect of the present invention is a pharmaceutical combination, preferably a cancer treatment combination, which includes a compound of formula (I″) and erlotinib. Alternatively, in another aspect of the present invention is a pharmaceutical combination, preferably a cancer treatment combination, which includes a compound of formula (I″), gefitinib, and erlotinib. The treatment combination may be a unitary pharmaceutical composition wherein each of the compound of formula (I″), gefitinib, and erlotinib (or combinations thereof) reside in the same composition or separate compositions wherein each active component is in an independent composition.

While it is possible that, for use in the cancer treatment methods of the present invention therapeutically effective amounts of a compound of formula (I″) and gefitinib and/or erlotinib as well as salts or solvates thereof, may be administered as the raw chemical, it is possible to present the active ingredient as a pharmaceutical composition. Accordingly, the invention further provides pharmaceutical compositions, which may be administered in the cancer treatment methods of the present invention. The pharmaceutical compositions include therapeutically effective amounts of a compound of formula (I″) and/or gefitinib, and/or erlotinib and salts or solvates thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The compound of formula (I″) or gefitinib or erlotinib may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

As indicated, therapeutically effective amounts of a specific compound of formula (I) or gefitinib or erlotinib are administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the compound of formula (I) will be given in the range of 0.1 to 100 mg/kg body weight of recipient (mammal) per day and more usually in the range of 1 to 10 mg/kg body weight per day.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

N (Normal) Kg (kilogram)

i. v. (intravenous); Hz (Hertz);

MHz (megahertz); mol (moles);

mmol (millimoles); RT (room temperature);

min (minutes); h (hours);

mp (melting point); TLC (thin layer chromatography);

Tᵣ (retention time); RP (reverse phase);

DCM (dichloromethane); DCE (dichloroethane);

DMF (N,N-dimethylformamide); HOAc (acetic acid);

TMSE (2-(trimethylsilyl)ethyl); TMS (trimethylsilyl);

TIPS (triisopropylsilyl); TBS (t-butyldimethylsilyl);

HPLC (high pressure liquid chromatography);

THF (tetrahydrofuran); DMSO (dimethylsulfoxide);

EtOAc (ethyl acetate); DME (1,2-dimethoxyethane);

EDTA ethylenediaminetetraacetic acid

FBS fetal bovine serum

IMDM Iscove's Modified Dulbecco's medium

PBS phosphate buffered saline

RPMI Roswell Park Memorial Institute

RIPA buffer *

RT room temperature *150 mM NaCl, 50 mM Tris-HCl, pH 7.5, 0.25% (w/v)-deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

GW572016F is lapatinib whose chemical name is N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate.

Gefitinib is Iressa© whose chemical name is 4-quinazolinamine,N-(3-chloro-4-fluorophenyl)-7-methoxy-6-[3-4-morpholin)propoxy].

Erlotinib is Tarceva™, whose chemical name is N-(3-ethynylphenyl)-6,7-bis(2-methoxyethoxy)-4-quinazolinamine; is commercially available as tablets as TARCEVA®.

### Example 1

### Preparation of GW572016F

### Stage 1

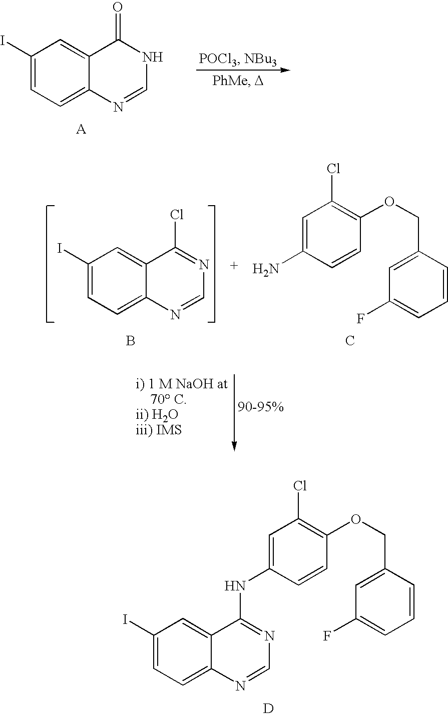

A stirred suspension of 3H-6-iodoquinazolin-4-one (compound A) in toluene (5 vols) was treated with tri-n-butylamine (1.2 eq.) at 20 to 25° C., then heated to 90° C. Phosphorous oxychloride (1.1 eq) was added, the reaction mixture was then heated to reflux. The reaction mixture was cooled to 50° C. and toluene (5 vols) added. Compound C (1.03 eq.) was added as a solid, the slurry was warmed back to 90° C. and stirred for 1 hour. The slurry was transferred to a second vessel; the first vessel was rinsed with toluene (2 vol) and combined with the reaction mixture. The reaction mixture was cooled to 70° C. and 1.0 M aqueous sodium hydroxide solution (16 vols) added dropwise over 1 hour to the stirred slurry maintaining the contents temperature between 68-72° C. The mixture was stirred at 65-70° C. for 1 hour and then cooled to 20° C. over 1 hour. The suspension was stirred at 20° C. for 2 hours, the product collected by filtration, and washed successively with water (3×5 vols) and ethanol (IMS, 2×5 vols), then dried in vacuo at 50-60° C.

Volumes are quoted with respect of the quantity of Compound A used. Percent yield range observed: 90 to 95% as white or yellow crystals.

### Stage 2

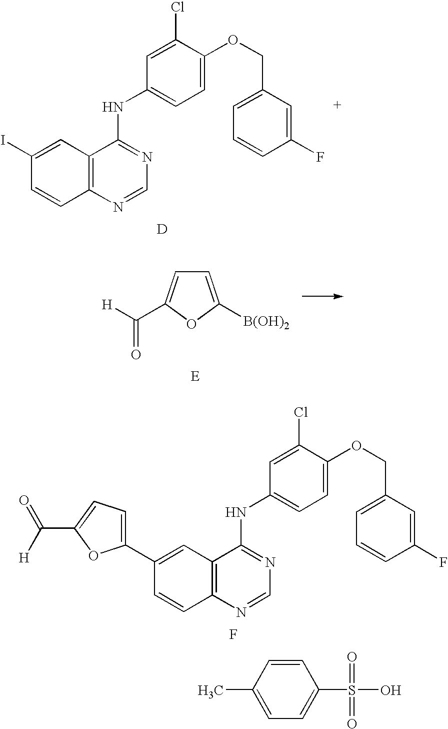

A mixture of N-{3-chloro-4-[(3-fluorobenzyl)oxy]phenyl}-6-iodo-4-quinazolinamine—compound D (1 wt), boronic acid—compound E (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then hot-filtered to remove the catalyst. The vessel was rinsed with IMS (2 vol).

A solution of p-toluenesulfonic acid monohydrate (1.5 wt, 4 equiv) in water (1.5 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the compound F as a yellow-orange crystalline solid (isolated as the ethanol solvate containing approximately 5% w/w EtOH).

### Stage 3

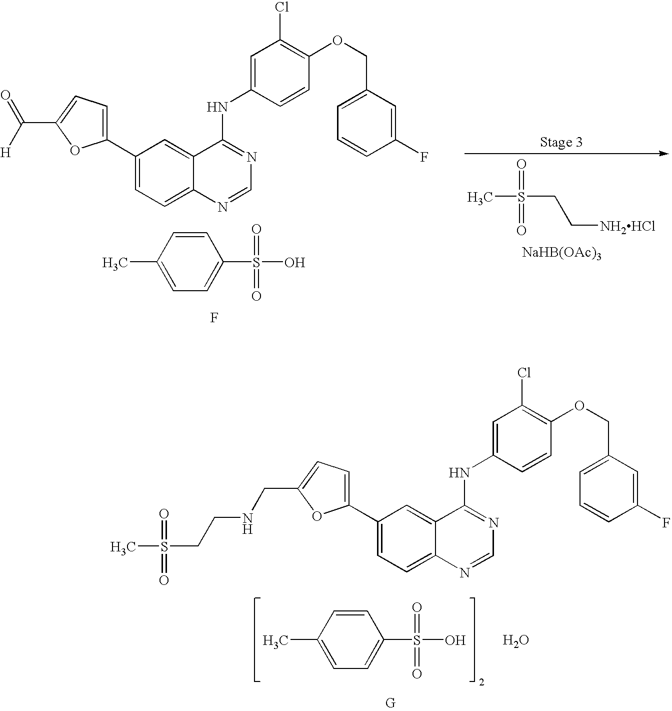

Compound F (1 wt) and 2-(methylsulfonyl)ethylamine hydrochloride (0.4 wt, 1.62 equiv.) were suspended in THF (10 vols). Sequentially, acetic acid (0.354 vol., 4 equiv.) and di-isopropylethylamine (DIPEA, 1.08 vol., 4.01 equiv.) were added. The resulting solution was stirred at 30°-35° C. for ca 1 hour then cooled to ca 22° C. Sodium tri-acetoxyborohydride (0.66 wt, 2.01 equiv.) was then added as a continual charge over approximately 15 minutes (some effervescence is seen at this point). The resulting mixture was stirred at ca 22° C. for ca 2 hours then sampled for HPLC analysis. The reaction was quenched by addition of aqueous sodium hydroxide (25% w/w, 3 vols.) followed by water (2 vols.) and stirred for ca 30 minutes (some effervescence was seen at the start of the caustic addition).

The aqueous phase was then separated, extracted with THF (2 vols) and the combined THF extracts were then washed twice with 25% w/v aqueous ammonium chloride solution (2×5 vols)². A solution of p-toluenesulfonic acid monohydrate (p-TSA, 0.74 wt, 2.5 equiv.) in water (1 vol)¹ was prepared, warmed to ca 60° C., and GW572016F (Compound G) (0.002 wt) seeds were added. ¹Minimum reaction volume ca 1 vol.²Maximum reaction volume ca 17 vol.

The THF solution of the free base of GW572016 was added to the p-TSA solution over at least 30 minutes, while maintaining the batch temperature at 60±3° C. The resulting suspension was stirred at ca 60° C. for 1-2 hours, cooled to 20-25° C. over an hour and aged at this temperature for ca 1 hr. The solid was collected by filtration, washed with 95:5 THF:Water (3×2 vols) and dried in vacuo at ca 35° C. to give GW572016F—compound G as a bright yellow crystalline solid. Expected yield 80% theory, 117% w/w.

### # Corrected for assay.

### Stage 4

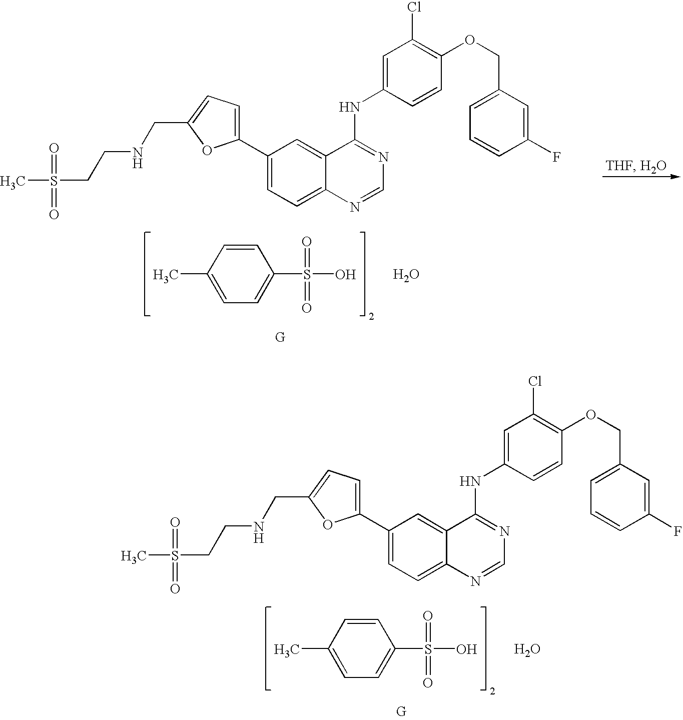

A suspension of the ditosylate monohydrate salt of N-{3-Chloro-4-[(3-fluorobenzyl)oxy]phenyl}-6-[5-({[2-(methane sulphonyl)ethyl]amino}methyl)-2-furyl]-4-quinazolinamine—compound G (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3 ratio, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2 vol) then tetrahydrofuran/water (19:1 ratio, 2 vol) and dried in vacuo at 45° C. to give N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate—compound G as a bright yellow crystalline solid.

### Example 2

### Preparation of Gefitinib

Gefitinib was prepared according to the procedures of International Patent Application No. PCT/GB96/00961, filed Apr. 23, 1996, and published as WO 96/33980 on Oct. 31, 1996 on pages 13-15 and at Example 1.

### Example 3

### Dosing with Lapatinib and Gefitinib

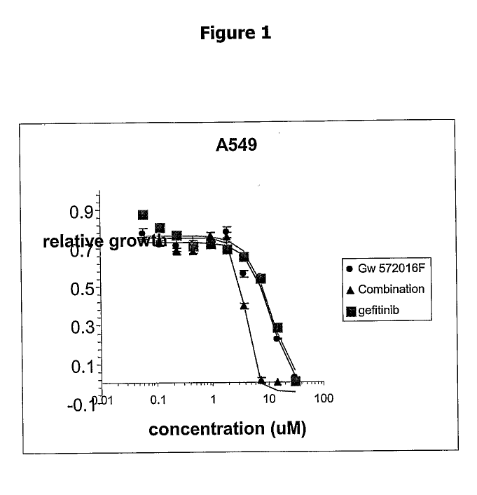

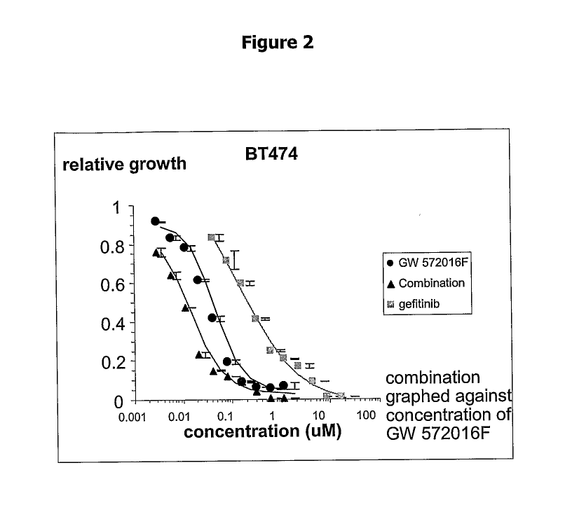

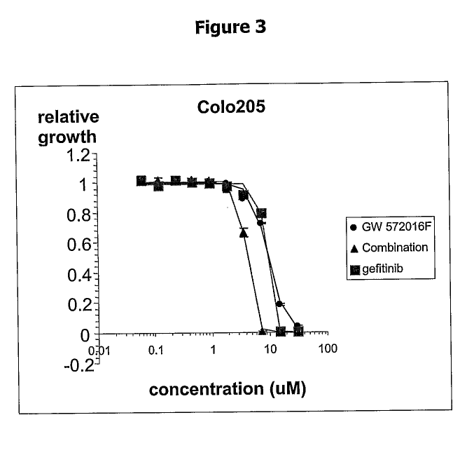

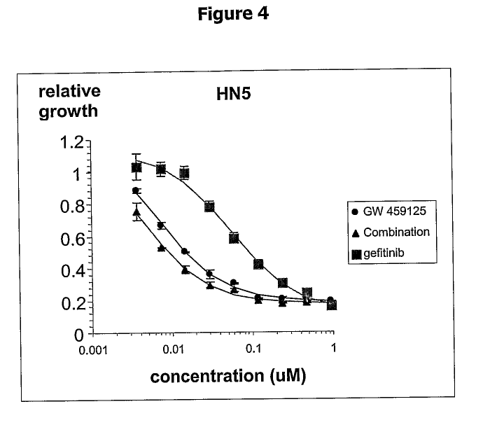

Cell lines were obtained from the American Type Culture Collection. The cells were maintained in tissue culture flasks in RPMI 1640 (Invitrogen #22400-089) with 10% fetal bovine serum (FBS, HyClone #SH30071.03) and were incubated at 37° Celsius in an atmosphere of 5% CO₂, until plating for IC₅₀ determination. For IC₅₀ determination, cells were plated in the appropriate medium at 5,000 cells per well in a 96-well tissue culture dish and returned to the incubator overnight. Approximately twenty-four hours after initial seeding, cells were exposed to the ditosylate salt form of GW 572016, GW 572016F alone, gefitinib alone, or GW 572016F and gefitinib in combination. Cells were dosed in 50% RPMI and 50% low glucose DMEM medium containing, 5% FBS, 50 micrograms/ml gentamicin and 0.6% DMSO. Dose concentration ranges were as reflected in the graphs of FIGS. 1-4. All dosing was performed concomitantly, and the dose ratio of each agent to GW 572016F was adjusted to approximately reflect the relative potency of each agent on each cell line. In most cases, the agents were dosed at a single fixed ratio.

After three days of compound exposure, the growth medium was removed by aspiration. Cell biomass was estimated by staining cells in 0.1 ml per well of methylene blue (Sigma #M9140, 0.5% in 50:50, ethanol:water), followed by incubation at room temperature for at least 30 minutes. Stain was aspirated and the plates rinsed by immersion in deionized water, followed by air drying. Stain was released from cells by the addition of 0.1 ml of solubilization solution (1.0% N-lauryl sarcosine, Sodium salt, Sigma #L5121 in PBS). Plates were incubated at room temperature for 40 minutes. Absorbance was read at 620 nM in a Tecan Spectra micro-plate reader. Percent inhibition of cell growth was calculated relative to untreated control wells. IC₅₀ values were interpolated using the method of Levenberg and Marquardt (Mager, 1972) and the equation: y=Vₘₐₓ*[1−(xⁿ/(Kⁿ+xⁿ))], where “K” is equal to IC₅₀.

IC50 values were generated for each agent individually and in combination. IC50 values were inserted into the combination index (CI) equation from Chou and Talalay:

D_{a,comb}/Dₐ+D_{b,comb}/D_{b}, where Dₐ and D_{b} are the IC50s of each agent alone. D_{a,comb} is the amount of agent a in the combination where the effect is 50% inhibition. D_{b,comb} is the amount of agent b in the combination where the effect is 50% inhibition. If the agents are dosed at a 1:1 ratio of each other, D_{a,comb}=D_{b,comb}. Values greater than 1 suggest antagonism. Values less than 1 suggest synergism. The extent of antagonism or synergism can be assumed to be reflected by the difference of the value from 1.0, ie 0.5 is more synergistic than 0.8 and 2.0 is more antagonistic than 1.5. It is important to note that the value 1.0 is predicted additivity. It is possible for a combination to give a greater inhibitory effect than either agent alone, but still be considered antogonisitic. This happens when the magnitude of combined effect is not as much as the mathematical model would predict. Another analysis template is being developed to compare the combination to the best single agent. The Chou and Talalay model also assumes that the individual agents are acting independently or on independent pathways and are mutually exclusive. Using a model that assumes the agents are working by the same mechanism as GW 572016F (mutually non-exclusive) increases some of the CI values, but does not change the ranking of the agents in this data set. The table below includes the combination index values for both mutually exclusive and mutually non-exclusive CI determination.

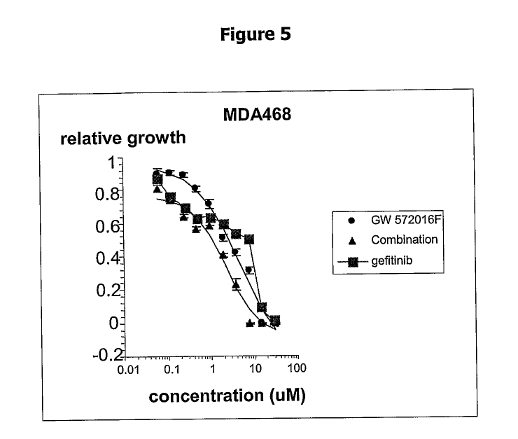

Results are depicted in Table 1 and FIGS. 1-5.

TABLE 1

| | | | CI* |
|---|---|---|---|
| | | CI* | Mutually Non- |
| Combination | Cell Line | Mutually Exclusive | exclusive |
| Lapatinib/gefitinib | A549 | 0.81 ± 0.14 | 0.98 ± 0.19 |
| Lapatinib/gefitinib | A549 | 0.81 ± 0.11 | 0.98 ± 0.15 |
| Lapatinib/gefitinib | Colo205 | 0.84 ± 0.04 | 1.01 ± 0.07 |
| Lapatinib/gefitinib | MDA468 | 0.71 ± 0.13 | 0.84 ± 0.17 |
| Lapatinib/gefitinib | MDA468 | 0.84 ± 0.09 | 0.98 ± 0.10 |
| Lapatinib/gefitinib | BT474 | 0.91 ± 0.04 | 1.1 ± 0.07 |
| Lapatinib/gefitinib | HN5 | 0.69 ± 0.07 | 0.74 ± 0.09 |
| Lapatinib/gefitinib | NCI-H1299 | 0.72 ± 0.23 | 0.85 ± 0.28 |
| Lapatinib/gefitinib | NCI-H1975 | 1.43 ± 0.12 | 1.84 ± 0.07 |

*Data is presented as an average of 3 experiments ± 95% confidence interval.

### NCI-H358 cells were run, however the obtained data was not suitable to fit a 4 parameter dose response curve.

### Example 4

### Preparation of Erlotinib

Erlotinib may be prepared according to procedures similar to those disclosed in U.S. Pat. No. 5,747,498 at column 22, lines 30-50 and at Example 20.

### Example 4

### Dosing with Lapatinib and Erlotinib

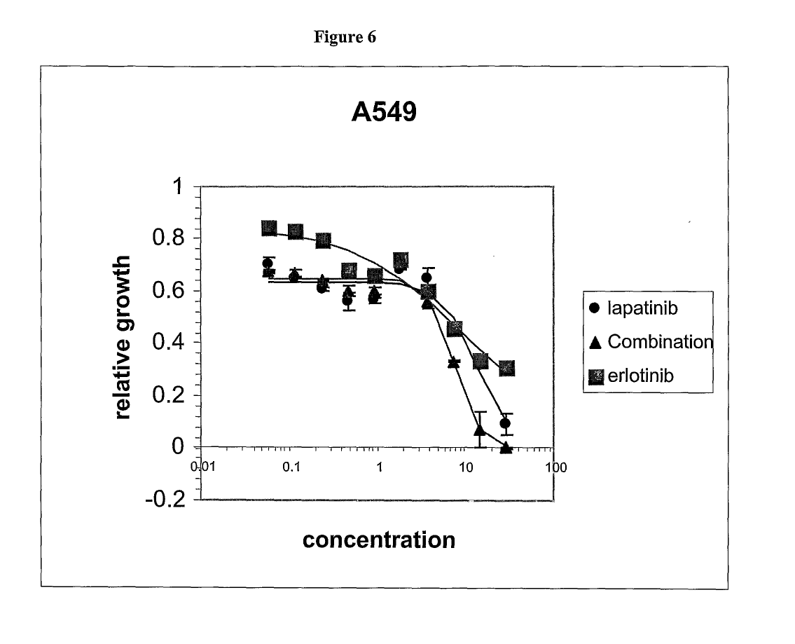

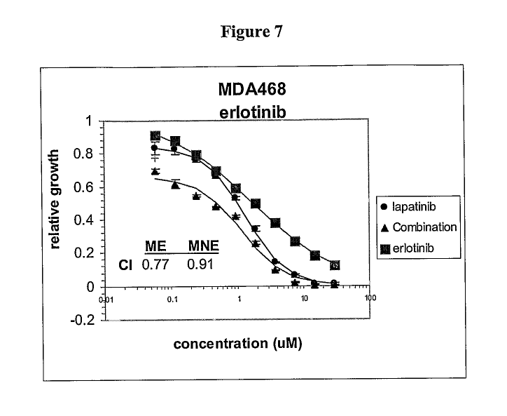

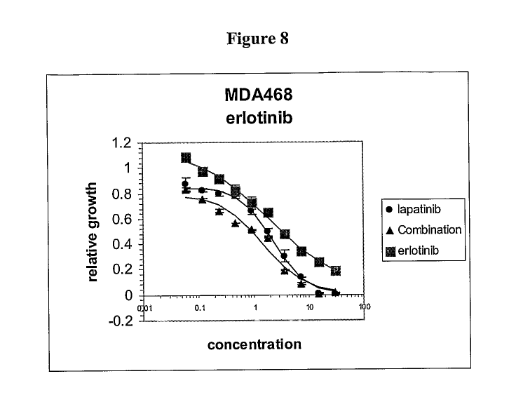

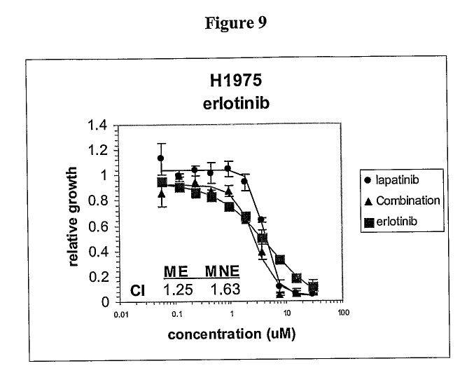

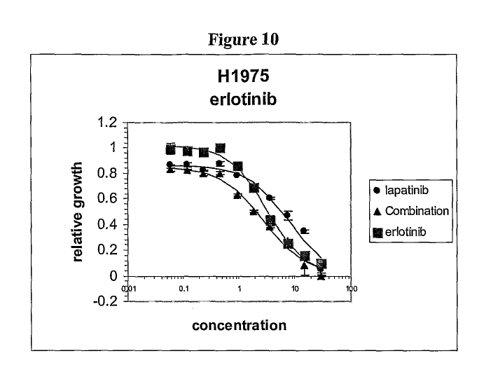

Cell lines were obtained from the American Type Culture Collection. The cells were maintained in tissue culture flasks in RPMI 1640 (Invitrogen #22400-089) with 10% fetal bovine serum (FBS, HyClone #SH30071.03) and were incubated at 37° Celsius in an atmosphere of 5% CO₂, until plating for IC₅₀ determination. For IC₅₀ determination, cells were plated in the appropriate medium at 5,000 cells per well in a 96-well tissue culture dish and returned to the incubator overnight. Approximately twenty-four hours after initial seeding, cells were exposed to the ditosylate salt form of GW 572016, GW 572016F alone, erlotinib alone, or GW 572016F and erlotinib in combination. Cells were dosed in 50% RPMI and 50% low glucose DMEM medium containing, 5% FBS, 50 micrograms/ml gentamicin and 0.6% DMSO. Dose concentration ranges were as reflected in the graphs of FIGS. 6-10. All dosing was performed concomitantly, and the dose ratio of each agent to GW 572016F was adjusted to approximately reflect the relative potency of each agent on each cell line. In most cases, the agents were dosed at a single fixed ratio.

After three days of compound exposure, the growth medium was removed by aspiration. Cell biomass was estimated by staining cells in 0.1 ml per well of methylene blue (Sigma #M9140, 0.5% in 50:50, ethanol:water), followed by incubation at room temperature for at least 30 minutes. Stain was aspirated and the plates rinsed by immersion in deionized water, followed by air drying. Stain was released from cells by the addition of 0.1 ml of solubilization solution (1.0% N-lauryl sarcosine, Sodium salt, Sigma #L5121 in PBS). Plates were incubated at room temperature for 40 minutes. Absorbance was read at 620 nM in a Tecan Spectra micro-plate reader. Percent inhibition of cell growth was calculated relative to untreated control wells. IC₅₀ values were interpolated using the method of Levenberg and Marquardt (Mager, 1972) and the equation: y=Vₘₐₓ*[1−(xⁿ/(Kⁿ+xⁿ))], where “K” is equal to IC₅₀.

IC50 values were generated for each agent individually and in combination. IC50 values were inserted into the combination index (CI) equation from Chou and Talalay as described above

### Results are depicted in Table 2 and FIGS. 6-10.

TABLE 2

| | | | CI* |
|---|---|---|---|
| | | CI* | Mutually Non- |
| Combination | Cell Line | Mutually Exclusive | exclusive |
| Lapatinib/erlotinib | A549 | 1.51 | 2.07 |
| Lapatinib/erlotinib | MDA468 | 0.77 | 0.91 |
| Lapatinib/erlotinib | MDA468 | 0.81 | 0.96 |
| Lapatinib/erlotinib | NCI-H1975 | 1.25 | 1.63 |
| Lapatinib/erlotinib | NCI-H1975 | 1.25 | 1.57 |

NCI-H358 cells were run, however the obtained data was not suitable to fit a 4 parameter dose response curve.

What is claimed is:

1. A method of treating breast cancer in a mammal, comprising: administering to said mammal therapeutically effective amounts of
(i) a compound of formula (I″) 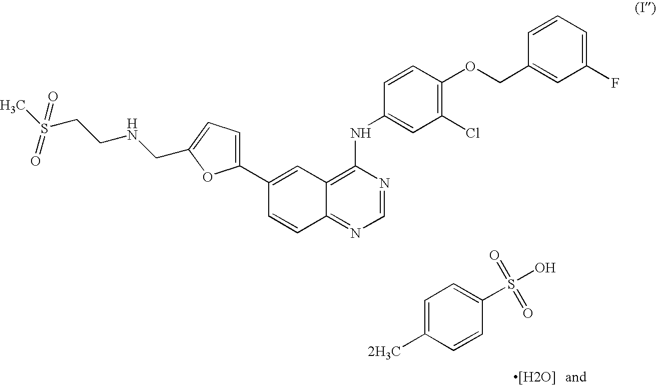
(ii) gefitinib or salts or solvates thereof.

2. A cancer treatment combination, comprising: therapeutically effective amounts of
(i) a compound of formula (I″) 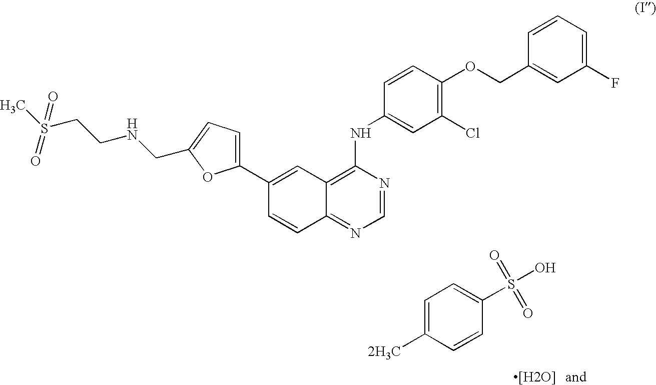
(ii) gefitinib or salts or solvates thereof.

3. A method of treating breast cancer in a mammal, comprising: administering to said mammal therapeutically effective amounts of
(i) a compound of formula (I″) 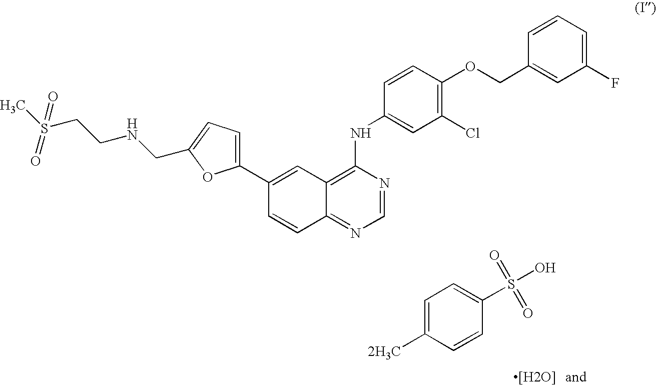
(ii) erlotinib or salts or solvates thereof.

4. A cancer treatment combination, comprising: therapeutically effective amounts of
(i) a compound of formula (I″) 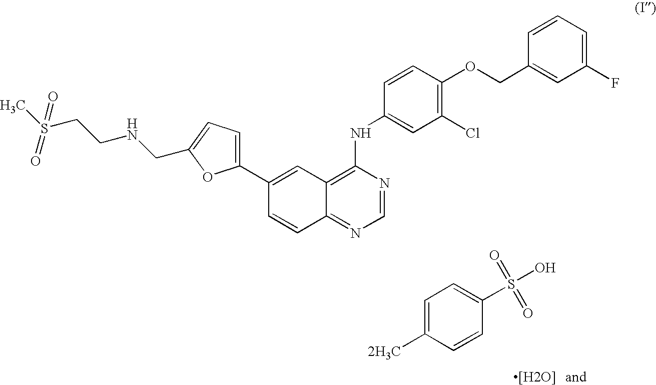
(ii) erlotinib or salts or solvates thereof.
